(19) United States
(12) Patent Application Publication
(10) Pub. No.: US 2013/0122051 A1
(43) Pub. Date: May 16, 2013

(54) Methods of preparing progesterone pharmaceutical compositions

(71) Applicants: Syed E. Abidi, Luthersville, MD (US); Eric R. Coutinho, Cockeysville, MD (US); Sridhar Gumudavelli, Cockeysville, MD (US); Swati Agrawal, Cockeysville, MD (US); Rampurna P. Gullapalli, Timonium, MD (US)

(72) Inventors: Syed E. Abidi, Luthersville, MD (US); Eric R. Coutinho, Cockeysville, MD (US); Sridhar Gumudavelli, Cockeysville, MD (US); Swati Agrawal, Cockeysville, MD (US); Rampurna P. Gullapalli, Timonium, MD (US)

(73) Assignee: PHARMACEUTICS INTERNATIONAL, INC., Hunt Valley, MD (US)

(21) Appl. No.: 13/481,397

(22) Filed: May 25, 2012

Related U.S. Application Data
(60) Provisional application No. 61/560,028, filed on Nov. 15, 2011.

(51) Int. Cl. A61K 31/57; A61P 15/00; A61P 15/06; A61K 9/14

(52) U.S. Cl. 424/400; 514/177

(57) ABSTRACT

The invention provides a method of preparing a pharmaceutical composition comprising: (a) combining progesterone particles with a liquid carrier to provide a mixture; (b) wet-milling the mixture to provide a wet-milled progesterone composition; and (c) processing the wet-milled progesterone composition to provide a pharmaceutical composition. Pharmaceutical compositions prepared by the method are also provided.

### CROSS-REFERENCE TO RELATED APPLICATION

This patent application claims the benefit of U.S. Provisional Patent Application No. 61/560,028, filed Nov. 15, 2011, which is incorporated herein by reference in its entirety.

### BACKGROUND OF THE INVENTION

Progesterone is useful for treating any of a variety of different conditions such as, for example, successive miscarriages, menstrual cycle disturbances, and premenstrual syndrome. Nevertheless, the development of methods for preparing stable progesterone pharmaceutical compositions poses challenges. Accordingly, there is a need for improved methods of preparing progesterone pharmaceutical compositions.

### BRIEF SUMMARY OF THE INVENTION

An embodiment of the invention provides a method of preparing a pharmaceutical composition comprising: (a) combining progesterone particles with a liquid carrier to provide a mixture; (b) wet-milling the mixture to provide a wet-milled progesterone composition; and (c) processing the wet-milled progesterone composition to provide a pharmaceutical composition.

Another embodiment of the invention provides a method of preparing a pharmaceutical composition comprising wet-milling progesterone particles in a liquid carrier to provide a wet-milled progesterone composition and processing the wet-milled progesterone composition to provide a pharmaceutical composition.

Still another embodiment of the invention provides a method of preparing a wet-milled progesterone composition comprising: (a) combining progesterone particles with a liquid carrier, optionally with at least one phospholipid and/or at least one lipophilic surfactant, to provide a mixture; and (b) wet-milling the mixture to provide a wet-milled progesterone composition.

Additional embodiments of the invention provide pharmaceutical compositions and wet-milled progesterone compositions prepared according to any of the methods described herein.

### BRIEF DESCRIPTION OF THE SEVERAL VIEWS OF THE DRAWING(S)

FIG. 1A is a graph showing the percent volume of PROMETRIUM capsules (circles ∘) or a second pass sample of a wet-milled progesterone composition prepared according to an embodiment of the method of the invention (asterisks *) having a given geometric diameter (microns).

FIG. 1B is a graph showing the cumulative percent volume of PROMETRIUM capsules (circles ∘) or a second pass sample of a wet-milled progesterone composition prepared according to an embodiment of the method of the invention (asterisks *) that is less than a given geometric diameter (microns).

FIGS. 2A and 2B are graphs showing the cumulative percent volume of second pass (2A) and third pass (2B) samples of a wet-milled progesterone composition having lecithin contents of 0% (circles ∘), 0.1% (dashes -), 0.2% (triangles Δ), 0.3% (squares □), or 0.4% (asterisks *), freshly prepared according to an embodiment of the method of the invention, that is less than a given geometric diameter (microns).

FIG. 3A is a graph showing the percent volume of a second pass sample of a wet-milled progesterone composition having 0.4% lecithin, which is prepared according to an embodiment of the method of the invention, and which is freshly prepared (initial) (open circles ∘) or which undergoes one (dashes -), two (closed circles ), three (squares □), or four (asterisks *) freeze/thaw cycles, having a given geometric diameter (microns).

FIG. 3B is a graph showing the cumulative percent volume of a second pass sample of a wet-milled progesterone composition having 0.4% lecithin, which is prepared according to an embodiment of the method of the invention, and which is freshly prepared (initial) (open circles ∘) or which undergoes one (dashes -), two (closed circles ), three (squares □),), or four (asterisks *) freeze/thaw cycles, that is less than a given geometric diameter (microns).

FIG. 4A is a graph showing the percent volume of PROMETRIUM capsules (circles ∘) or a second pass sample of a freshly prepared wet-milled progesterone composition having 0.4% lecithin and which is prepared according to an embodiment of the method of the invention (asterisks *) having a given geometric diameter (microns).

FIG. 4B is a graph showing the cumulative percent volume of PROMETRIUM capsules (circles ∘) or a second pass sample of a freshly prepared wet-milled progesterone composition having 0.4% lecithin and which is prepared according to an embodiment of the method of the invention (asterisks *) that is less than a given geometric diameter (microns).

FIG. 5A is a graph showing the percent volume of PROMETRIUM capsules (circles ∘) or a third pass sample of a freshly prepared wet-milled progesterone composition having 0.4% lecithin and which is prepared according to an embodiment of the method of the invention (asterisks *) having a given geometric diameter (microns).

FIG. 5B is a graph showing the cumulative percent volume of PROMETRIUM capsules (circles ∘) or a third pass sample of a freshly prepared wet-milled progesterone composition having 0.4% lecithin and which is prepared according to an embodiment of the method of the invention (asterisks *) that is less than a given geometric diameter (microns).

FIG. 6A is a graph showing the percent volume of PROMETRIUM capsules (circles ∘) after two freeze/thaw cycles or a second pass sample of a wet-milled progesterone composition having 0.4% lecithin which is prepared according to an embodiment of the method of the invention (asterisks *) after four freeze/thaw cycles having a given geometric diameter (microns).

FIG. 6B is a graph showing the cumulative percent volume of PROMETRIUM capsules (circles ∘) after two freeze/thaw cycles or a second pass sample of a wet-milled progesterone composition having 0.4% lecithin and which is prepared according to an embodiment of the method of the invention (asterisks *) after four freeze/thaw cycles that is less than a given geometric diameter (microns).

FIG. 7A is a graph showing the percent volume of PROMETRIUM capsules (circles ∘) or a first pass sample of a wet-milled progesterone composition prepared according to an embodiment of the method of the invention before (asterisks *) or after (triangles Δ) encapsulation having a given geometric diameter (microns).

FIG. 7B is a graph showing the cumulative percent volume of PROMETRIUM capsules (circles ∘) or a first pass sample of a wet-milled progesterone composition prepared according to an embodiment of the method of the invention before (asterisks *) or after (triangles Δ) encapsulation that is less than a given geometric diameter (microns).

FIG. 8A is a graph showing the percent volume of PROMETRIUM capsules (circles ∘) or a second pass sample of a wet-milled progesterone composition prepared according to an embodiment of the method of the invention before (asterisks *) or after (triangles Δ) encapsulation having a given geometric diameter (microns).

FIG. 8B is a graph showing the cumulative percent volume of PROMETRIUM capsules (circles ∘) or a second pass sample of a wet-milled progesterone composition prepared according to an embodiment of the method of the invention before (asterisks *) or after (triangles Δ) encapsulation that is less than a given geometric diameter (microns).

### DETAILED DESCRIPTION OF THE INVENTION

An embodiment of the invention provides a method of preparing a pharmaceutical composition comprising: (a) combining progesterone particles with a liquid carrier to provide a mixture; (b) wet-milling the mixture to provide a wet-milled progesterone composition; and (c) processing the wet-milled progesterone composition to provide a pharmaceutical composition.

Progesterone is a steroid hormone and may be chemically described as pregn-4-ene-3,20-dione. Progesterone can be secreted by the body or chemically synthesized. Progesterone has the following chemical structure:

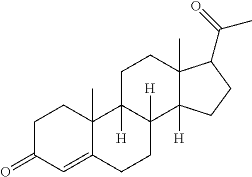

An embodiment of the inventive method may comprise combining a therapeutically effective amount of progesterone with the liquid carrier. The term “effective amount” or “therapeutically effective amount,” as used herein, refers to the amount of progesterone that is effective to achieve its intended purpose after a single dose, wherein a single dose comprises one or more dosage units, or after a course of doses, e.g., during or at the end of the treatment period. Thus, for example, the term “therapeutically effective amount” of the progesterone, when used in a method of treating endometrial hyperplasia, refers to that dose of progesterone that lessens or prevents the occurrence of endometrial hyperplasia when administered to a patient in need of such treatment. The therapeutically effective amount will vary depending on the needs of the patient, but this amount can readily be determined by one of skill in the art, for example, a physician.

The inventive method may comprise combining any therapeutically effective amount of progesterone with the liquid carrier. In some embodiments, the progesterone is present in an amount ranging from about 4% or less to about 80% or more by weight of the mixture. In some embodiments, the progesterone is present in an amount ranging from about 10% to about 70% by weight of the mixture. In some embodiments, the progesterone is present in an amount ranging from about 20% to about 60% by weight of the mixture. In some embodiments, the progesterone is present in an amount ranging from about 40% to about 50% by weight of the mixture.

An embodiment of the inventive method may comprise combining any suitable dose of progesterone with the liquid carrier. In some embodiments, the mixture contains a dose of about 10 mg or less to about 500 mg or more of progesterone. In some embodiments, the mixture contains a dose of about 50 mg of progesterone to about 300 mg of progesterone. In some embodiments, the mixture contains a dose of about 100 mg, about 200 mg, about 300 mg, about 400 mg, or about 500 mg of progesterone.

The inventive method may comprise combining any suitable progesterone with the liquid carrier. The progesterone may comprise, for example, micronized progesterone particles or unmicronized progesterone particles. As used herein, the term “unmicronized progesterone particles” means a progesterone compound in particulate form in which less than about 1% of the particles have a particle size of less than about 60 microns. In a preferred embodiment, the progesterone particles are unmicronized.

The inventive method may comprise combining progesterone with any suitable liquid carrier. In some embodiments, the liquid carrier may comprise any one or more of a long-chain glyceride, a free fatty acid, a fatty acid ester, and a medium-chain glyceride.

In some embodiments, the liquid carrier comprises at least one long-chain glyceride. The long-chain glyceride may be any suitable long-chain glyceride including, but not limited to, any one or more of peanut oil, soybean oil, sunflower oil, olive oil, sesame oil, colza oil, almond oil, safflower oil, corn oil, linseed oil, rapeseed oil, evening primrose oil, grape seed oil, cottonseed oil, flaxseed oil, and menhaden oil. As used herein, “long chain glyceride” is any suitable glyceride having a chain of about 12 to about 20 carbons.

In some embodiments, the liquid carrier comprises at least one free fatty acid. The free fatty acid may be saturated or unsaturated, and may be any suitable free fatty acid including, but not limited to, myristoleic acid, palmitoleic acid, sapienic acid, oleic acid, elaidic acid, vaccenic acid, linoleic acid, linoelaidic acid, α-linolenic acid, arachidonic acid, eicosapentaenoic acid, erucic acid, docosahexaenoic acid, caprylic acid, capric acid, caproic acid, lauric acid, myristic acid, palmitic acid, stearic acid, arachidic acid, behenic acid, lignoceric acid, and cerotic acid.

In some embodiments, the liquid carrier comprises at least one fatty acid ester. The fatty acid ester may be saturated or unsaturated, and may be any suitable fatty acid ester including, but not limited to, a glycerol fatty acid ester, a propylene glycol fatty acid ester, a propyl alcohol fatty acid ester, and an ethanol fatty acid ester. The glycerol fatty acid ester may be any suitable glycerol fatty acid ester and may include, for example, any one or more of glycerol mono-, di-, or tri-esters of long chain or medium chain fatty acids. Exemplary glycerol fatty acid esters suitable for use in the inventive methods include, but are not limited to, glyceryl monooleate, and glyceryl monolinoleate. The propylene glycol fatty acid ester may be any suitable propylene glycol fatty acid ester and may include, for example, any one or more of propylene glycol mono- or di-esters of long chain or medium chain fatty acids. Exemplary propylene glycol fatty acid esters suitable for use in the inventive methods include, but are not limited to, propylene glycol monolaurate (e.g., LAUROGLYCOL propylene glycol monolaurate available from Gattefossé, Saint-Priest, France), propylene glycol monocaprylate, propylene glycol caprylate, propylene glycol dicaprylocaprate, and propylene glycol laurate. Exemplary ethanol fatty acid esters suitable for use in the inventive methods include, but are not limited to, ethyl oleate, ethyl palmitate, ethyl caprylate, ethyl myristate, and ethanol esters of any of the fatty acids described herein. Other exemplary fatty acid esters suitable for use in the inventive methods include, but are not limited to, polyethoxylated fatty acid esters, polyethylene glycol monostearate, polyoxyl stearate, polyethylene glycol hydroxystearate, and macrogol hydroxystearate. The propyl alcohol fatty acid ester may be any suitable propyl alcohol fatty acid ester. Exemplary propyl alcohol fatty acid esters include, but are not limited to, isopropyl myristate and isopropyl palmitate. As used herein, “long chain fatty acid” is any suitable fatty acid having a chain of about 12 to about 20 carbons including, for example, any of the long chain fatty acids described herein. As used herein, “medium chain fatty acid” is any suitable fatty acid having a chain of about 6 to about 12 carbons including, for example, any of the medium chain fatty acids described herein.

In some embodiments, the liquid carrier comprises at least one medium chain glyceride. As used herein, “medium chain glyceride” is any suitable glyceride having a chain of about 6 to about 12 carbons. Exemplary medium-chain glycerides suitable for use in the inventive methods include, but are not limited to, coconut oil (e.g., mono-, di-, and tri-glycerides of coconut oil), and palm oil.

In some embodiments, the liquid carrier comprises at least one phospholipid. The phospholipid may be any suitable phospholipid. Exemplary phospholipids suitable for use in the inventive methods include, but are not limited to, lecithin, sphingosylphosphocholine, 2-aminoglycerol-phosphocholine, serine-phosphocholine, threonine-phosphocholine, tyrosine-phosphocholine, aminoethanol-phosphocholine, hydroxyproline-phosphocholine, and sphingosyl-phosphocholine. Preferably, the phospholipid is lecithin. The lecithin may be any suitable type of lecithin. In an embodiment of the invention, the mixture comprises soy lecithin. The mixture may comprise any suitable amount of lecithin. In some embodiments, the mixture may comprise from about 0% to about 1.0% lecithin. In some embodiments, the mixture may comprise from about 0% to about 0.4% lecithin. In some embodiments, the mixture may comprise about 0% lecithin, about 0.1% lecithin, about 0.2% lecithin, about 0.3% lecithin, or about 0.4% lecithin. In an embodiment of the invention, the lecithin may be added to the mixture prior to wet-milling and/or may be added to the wet-milled progesterone composition after wet-milling (e.g., prior to processing to form a pharmaceutical composition).

In some embodiments, the liquid carrier comprises at least one lipophilic surfactant. The lipophilic surfactant may be any suitable type of lipophlic surfactant. Exemplary lipophilic surfactants suitable for use in the inventive methods include, but are not limited to, sorbitan monooleate, sorbitan trioleate, polyethylene glycol oleyl ethers, polyoxyethylene (2) oleyl ethers, polyoxyethylene (2) isooctylphenyl ethers, polyoxyethylene (2) octylphenyl ethers, sorbitan monopalmitate, sorbitan trioleate, sorbitan tristearate, sorbitan sesquioleate, sorbitan monooleate, sorbitan monostearate, ethylenediamine tetrakis(ethoxylate-block-propoxylate) tetrol, ethylenediamine tetrakis(propoxylate-block-ethoxylate) tetrol, poly(ethylene glycol)-block-poly(propylene glycol)-block-poly(ethylene glycol), poly(ethylene glycol)-block-poly(propylene glycol)-block-poly(ethylene glycol), poly(ethylene glycol)-block-poly(propylene glycol)-block-poly(ethylene glycol), poly(ethylene glycol)-block-poly(propylene glycol)-block-poly(ethylene glycol), poly(propylene glycol)-block-poly(ethylene glycol)-block-poly(propylene glycol), and glyceryl monostearate.

The amount of phospholipid and/or lipophilic surfactant by weight of the mixture may be adjusted, as appropriate. In some embodiments, the mixture comprises from about 0.05% to about 5% phospholipid and/or lipophilic surfactant by weight of the mixture.

In an embodiment of the invention, the liquid carrier is aqueous. The aqueous liquid carrier may comprise at least one stabilizer. The stabilizer may be any suitable stabilizer, including but not limited to lecithin and polysorbate. Examples of polysorbate include, but are not limited to, TWEEN 80 polysorbate and TWEEN 20 polysorbate.

The inventive method may comprise combining progesterone with any suitable amount of liquid carrier. In some embodiments, the liquid carrier is present in an amount ranging from about 20% to about 96% by weight of the mixture. In some embodiments, the liquid carrier is present in an amount ranging from about 30% to about 90% by weight of the mixture. In some embodiments, the amount of liquid carrier is present in an amount ranging from about 40% to about 80% by weight of the mixture. In some embodiments, the amount of liquid carrier is present in an amount ranging from about 50% to about 60% by weight of the mixture.

The inventive method may comprise combining progesterone with the liquid carrier in any suitable manner. In an embodiment of the invention, the method comprises suspending the progesterone in the liquid carrier. Accordingly, in some embodiments, the mixture may be a suspension.

In some embodiments, the pharmaceutical compositions prepared by the inventive methods may, optionally, comprise any one or more suitable excipients. In this regard, the mixture may, optionally, further comprise a disintegrant. As used herein, a “disintegrant” is a substance that has the ability to absorb oil or lipid materials to maintain the free-flowing property of the formulation despite a high percentage of low melting point oils or lipids in the formulation. Disintegrants include, but are not limited to, starches, clays, celluloses, algins, gums, and cross-linked polymers, including, e.g., crospovidone, sodium starch glycolate, croscarmellose, methylcellulose, agar, bentonite, cellulose and wood products, natural sponge, cation-exchange resins, alginic acid, guar gum, citrus pulp, carboxymethylcellulose, and combinations thereof.

In some embodiments, the mixture may, optionally, further comprise an absorbant. Suitable absorbants include, but are not limited to, SYLOID absorbant (W.R. Grace & Co., Columbia, Md.), silicon dioxide and its derivatives, micronized silicas, lactose, lactose monohydrate, methylcellulose, microcrystalline cellulose, sugars, maltodextrin, and mixtures thereof.

In some embodiments, the mixture may, optionally, further comprise an antioxidant. Suitable antioxidants include, but are not limited to, adipic acid, alpha lipoic acid, ascorbyl palmitate, biotin, boron, butylated hydroxyl toluene, butylated hydroxyanisole, carotenoids, calcium citrate, sodium metabisulfate, tocopherols, and mixtures thereof.

In some embodiments, the mixture may, optionally, further comprise a lubricant. Suitable lubricants include, but are not limited to, magnesium stearate, colloidal silicon dioxide, silica gel, aluminum stearate, talc, stearic acid, sodium stearate, calcium stearate, sodium stearyl fumarate, and mixtures thereof.

An embodiment of the inventive method comprises wet-milling the mixture to provide a wet-milled progesterone composition. “Wet-milling” can also be referred to as “media milling” or “wet-bead milling.” In an embodiment of the invention, the method comprises wet-milling the mixture in any suitable manner. Exemplary mills that may be suitable for wet-milling include, but are not limited to, ball (or bead) mill, rod mill, hammer mill, colloid mill, fluid-energy mill, high-speed mechanical screen mill, and centrifugal classifier mill. A preferred mill is the DYNOMILL mill (Glen Mills Inc., Clifton, N.J.). The size and amount of milling media (e.g., beads) may be varied, as appropriate, depending on, e.g., the desired size of the progesterone particles and the duration of the milling. In some embodiments, the milling media (e.g., beads) may be from about 0.5 mm to about 10 mm. The method may comprise wet-milling using any suitable amount of milling media. In some embodiments, the milling media may comprise from about 30% to about 70% of the volume of the mill chamber.

The inventive method may comprise wet-milling the mixture for any suitable duration. The duration of the wet-milling may be varied, as appropriate, depending on, e.g., the desired size of the progesterone particles, the size and/or amount of beads, and/or batch size. In some embodiments of the invention, the duration of the wet-milling may be from about one minute or less to about 20 minutes or more. In some embodiments, the duration of the wet-milling may be from about 2 minutes to about 15 minutes. In an embodiment of the invention, a change in any one or more of milling speed (impeller/tip speed), size or amount of the milling media, rate the mixture is fed into the mill, the viscosity or temperature of the mixture, amount of progesterone in the mixture, and size or hardness of progesterone particles may change the duration of milling required to achieve the desired particle size.

In embodiments which include wet-milling a mixture of progesterone and aqueous liquid carrier, the method comprises drying the wet-milled, progesterone composition having the desired progesterone particle size. The drying may be carried out in any suitable manner, including but not limited to, spray-drying. An embodiment of the method further comprises processing the wet-milled progesterone composition into any suitable pharmaceutical composition including, but not limited to, a tablet or a hard-shelled capsule. Another embodiment of the method further comprises processing the wet-milled progesterone composition by suspending the dried, wet-milled, progesterone composition in a lipophilic carrier including, but not limited to, any of the long-chain glycerides, free fatty acids, fatty acid esters, and medium-chain glycerides described herein. In this regard, the method further comprises processing the wet-milled progesterone composition into any suitable pharmaceutical composition including, but not limited to, a soft-shelled capsule.

The inventive method may comprise deaerating the wet-milled progesterone composition. Deaerating is optional and in some embodiments, the method may lack a deaerating step. Deaerating may be performed in any suitable manner such as, e.g., by vacuuming the mixture.

In an embodiment of the invention, deaerating the wet-milled progesterone composition provides a first-pass, wet-milled progesterone composition. A “pass,” as used herein, comprises wet-milling once and deaerating once as described herein. The inventive methods may comprise any suitable number of passes. The number of passes is not limited and in some embodiments, the inventive methods may comprise one, two, three, four, five, six, seven, eight, nine, ten, or more passes. In this regard, the inventive method may comprise repeating the wet-milling and/or deaerating described herein one or more times. The number of passes may be varied, as appropriate, depending on the desired size of the progesterone particles, the starting size of the progesterone particles, the amount of progesterone in the mixture, the amount of liquid carrier, the rate at which the mixture is added to the mill, and/or the temperature of the milling chamber. In some embodiments, the method comprises sizing a sample of the wet-milled, progesterone composition following each pass to determine if the progesterone particles have the desired size range. If the progesterone particles are too large, the method may comprise repeating wet-milling for one or more additional passes. If the progesterone particles have an acceptable size, the method may comprise processing the wet-milled progesterone composition to provide a pharmaceutical composition.

The wet-milling of the inventive method, regardless of the number of passes, may provide progesterone particles having any suitable cumulative size distribution. For example, the wet-milling may provide progesterone particles having any suitable cumulative size distribution D₉₀. In some embodiments, wet-milling provides progesterone particles having a cumulative size distribution D₉₀ from about 10 μm to about 150 μm. In some embodiments, wet-milling provides progesterone particles having a cumulative size distribution D₉₀ from about 10 μm to about 100 μm. In some embodiments, wet-milling provides progesterone particles having a cumulative size distribution D₉₀ from about 10 μm to about 90 μm. Preferably, wet-milling provides progesterone particles having a cumulative size distribution D₉₀ from about 10 μm to about 70 μm.

The wet-milling may provide progesterone particles having any suitable cumulative size distribution D₅₀. In some embodiments, wet-milling provides progesterone particles having a cumulative size distribution D₅₀ from about 5 μm to about 70 μm. In some embodiments, wet-milling provides progesterone particles having a cumulative size distribution D₅₀ from about 5 μm to about 60 μm. In some embodiments, wet-milling provides progesterone particles having a cumulative size distribution D₅₀ from about 5 μm to about 50 μm. Preferably, wet-milling provides progesterone particles having a cumulative size distribution D₅₀ from about 5 μm to about 30 μm.

The wet-milling may provide progesterone particles having any suitable cumulative size distribution D₁₀. In some embodiments, wet-milling provides progesterone particles having a cumulative size distribution D₁₀ from about 2 μm to about 35 μm. In some embodiments, wet-milling provides progesterone particles having a cumulative size distribution D₁₀ from about 2 μm to about 30 μm. In some embodiments, wet-milling provides progesterone particles having a cumulative size distribution D₁₀ from about 2 μm to about 20 μm. Preferably, wet-milling provides progesterone particles having a cumulative size distribution D₁₀ from about 2 μm to about 15 μm.

The wet-milling may provide progesterone particles having any suitable cumulative size distribution D_{4,3}. In some embodiments, wet-milling provides progesterone particles having a cumulative size distribution D_{4,3} from about 9 μm to about 80 μm. In some embodiments, wet-milling provides progesterone particles having a cumulative size distribution D_{4,3} from about 12 μm to about 50 μm. In some embodiments, wet-milling provides progesterone particles having a cumulative size distribution D_{4,3} from about 15 μm to about 40 μm. Preferably, wet-milling provides progesterone particles having a cumulative size distribution D_{4,3} from about 18 μm to about 29 μm.

Any of the cumulative size distributions described herein may have any suitable span. In some embodiments, the span is from about 1.0 μm to about 3.0 μm. In some embodiments, the span is from about 1.2 μm to about 2.5 μm. In some embodiments, the span is from about 1.3 μm to about 2.0 μm. Preferably, the span is from about 1.45 μm to about 1.54 μM.

The wet-milling may provide a wet-milled progesterone composition having any suitable viscosity. In some embodiments, wet-milling provides a wet-milled progesterone composition having a viscosity from about 500 cP to about 8,000 cP. In some embodiments, wet-milling provides a wet-milled progesterone composition having a viscosity from about 900 cP to about 7,000 cP. In some embodiments, wet-milling provides a wet-milled progesterone composition having a viscosity from about 1,400 cP to about 6,000 cP. Preferably, wet-milling provides a wet-milled progesterone composition having a viscosity from about 1,400 cP to about 5,000 cP.

The pharmaceutical composition prepared by the inventive methods may be in any suitable dosage form. In some embodiments, the pharmaceutical composition is an oral pharmaceutical composition. The oral pharmaceutical composition may be in any suitable form, for example, a pill, tablet, or capsule. Preferably, the pharmaceutical composition is a capsule. The capsule may be a hard-shelled capsule or a soft-shelled capsule. In an especially preferred embodiment, the pharmaceutical composition is a soft-shelled capsule.

In some embodiments, the soft-shelled capsule may be a soft, globular shell that may be thicker than the shell of hard gelatin capsules. The soft-shell may comprise gelatin. The soft-shell may further comprise plasticizers such as, for example, glycerin, sorbitol, or a similar polyol. These capsules may be sealed at a seam to avoid premature breakage. The shell may further comprise additional components such as, for example, water, titanium dioxide, flavor, sweetener, enteric polymer, non-gelatin film former, and/or dye.

The capsule may have any suitable size. These sizes range from about 000 to about 5 for hard shelled capsules and from about 1 to about 480 for soft shell capsules (also referred to as softgels, soft elastic capsules, or soft gelatin capsules) as described in Remington: The Science and Practice of Pharmacy, Lippincott Williams & Wilkins, 19th ed. (1995) (hereinafter Remington's) and The Theory and Practice of Industrial Pharmacy, Lea & Febiger, Third Edition (1986). The appropriate capsule size may be readily determined by one of skill in the art depending on the amount and volume of progesterone composition, e.g. the number of milligrams and volume of progesterone in the capsule, to be delivered to the patient.

The pharmaceutical compositions prepared by the inventive methods may be used to treat or prevent any of a variety of different conditions. Such conditions may include, but are not limited to, endometriosis, luteal phase deficiency, successive miscarriages, menstrual cycle disturbances (e.g., secondary amenorrhea, irregular bleeding), premenstrual syndrome, preterm delivery, and endometrial hyperplasia. Additionally, the pharmaceutical compositions prepared by the inventive methods may be useful in contraceptive methods, to reduce risk of endometrial cancer, or to assist in reproductive techniques such as, for example, in vitro fertilization.

An embodiment of the inventive method comprises processing the wet-milled progesterone composition to provide a pharmaceutical composition. The processing of the inventive method may be in any suitable manner to provide any suitable dosage form. In some embodiments, processing the wet-milled progesterone composition comprises encapsulating the wet-milled progesterone composition to provide a capsule. The pharmaceutical compositions prepared by the methods of the present invention can be encapsulated using large-scale production methods. Suitable methods of encapsulation include plate processes, rotary die-processes, microencapsulation processes, and machine encapsulation processes as disclosed in Remington's.

Another embodiment of the invention provides a method of preparing a pharmaceutical composition comprising wet-milling progesterone particles in a liquid carrier to provide a wet-milled progesterone composition and processing the wet-milled progesterone composition to provide a pharmaceutical composition. The method comprises wet-milling and processing as described herein with respect to other aspects of the invention.

Another embodiment of the invention provides a pharmaceutical composition prepared according to any of the methods described herein.

Another embodiment of the invention provides a method of preparing a wet-milled progesterone composition comprising: (a) combining progesterone particles with a liquid carrier, optionally with at least one phospholipid and/or at least one lipophilic surfactant, to provide a mixture; and (b) wet-milling the mixture to provide a wet-milled progesterone composition. “Wet-milling,” as used herein, means milling the progesterone particles in any suitable liquid carrier as described herein. The method comprises combining and wet-milling as described herein with respect to other aspects of the invention.

Another embodiment of the invention provides a wet-milled progesterone composition prepared according to any of the methods described herein.

The following examples further illustrate the invention but, of course, should not be construed as in any way limiting its scope.

### Example 1

This example demonstrates the particle size of a wet-milled, progesterone composition prepared according to an embodiment of the method of the invention.

A suspension is prepared comprising unmicronized (D₉₀ 220 μm) progesterone (40.0% w/w), peanut oil (59.6% w/w), and soy lecithin (0.4% w/w). In a first pass, the suspension is wet-milled in a 0.6 L (milling chamber volume) DYNOMILL mill (Glen Mills Inc., Clifton, N.J.) at 2,500 revolutions per minute (rpm) using 1.5 mm very high density zirconium oxide beads and deaerated to provide a first pass, wet-milled progesterone composition. In a second pass, the first pass, wet-milled progesterone composition is milled and deaerated as described above to provide a second pass, wet-milled progesterone composition. In a third pass, the second pass, wet-milled progesterone composition is milled and deaerated as described above to provide a third pass, wet-milled progesterone composition. The milling conditions, including percent beads, input, and output, are varied for each of the first, second, and third passes as set forth in Table 1.

Samples of each of the first, second, and third pass wet-milled progesterone compositions prepared under the various milling conditions are collected and the particle size (μm) is measured using a MALVERN MASTERSIZER particle size analyzer (Malvern Instruments, Westborough Mass.). Saturated, filtered, progesterone in sunflower oil is used as the diluent. The results are shown in Table 1. “Initial” refers to unmilled progesterone suspension. “% Beads” refers to the volume of milling chamber filled with milling media (beads). “Input” refers to the pump setting. “Output” refers to the product (gm/min) received from the mill.

TABLE 1

| Lot # | % Beads | Pump Type | Input | Output | Pass# | D10 | D50 | D90 | D (4, 3) | Span |
|---|---|---|---|---|---|---|---|---|---|---|
| 10Pii0297-039 | 65% | Peristaltic | 430 mL/min | — | Initial | 12.023 | 78.103 | 210.546 | 96.559 | 2.542 |
| | | | | 284 | 1 | 6.051 | 16.551 | 53.426 | 27.729 | 2.862 |
| | | | | 73 | 2 | 5.000 | 9.631 | 18.766 | 10.942 | 1.429 |
| | | | | 21 | 3 | 4.736 | 9.064 | 17.593 | 10.252 | 1.419 |
| 10Pii0297-043 | 50% | Peristaltic | 430 mL/min | — | Initial | 12.023 | 78.103 | 210.546 | 96.559 | 2.542 |
| | | | | 249 | 1 | 7.076 | 22.109 | 72.290 | 35.202 | 2.950 |
| | | | | 147 | 2 | 5.898 | 13.532 | 31.893 | 16.752 | 1.921 |
| | | | | 72 | 3 | 5.665 | 12.015 | 25.823 | 14.246 | 1.678 |
| | | | Particle Size Stability | | Pass 2 after 2 wks | 7.103 | 17.606 | 39.719 | 20.993 | 1.853 |
| | | | | | Pass 3 after 2 wks | 6.272 | 14.260 | 30.495 | 16.601 | 1.699 |
| 10Pii0297-052 | 50% | Peristaltic | — | — | Initial | 12.023 | 78.103 | 210.546 | 96.559 | 2.542 |
| | | | 430 mL/min | 205 | 1 | 7.239 | 21.799 | 64.949 | 33.076 | 2.647 |
| | | | 550 mL/min | 195 | 2 | 7.257 | 21.406 | 62.877 | 30.845 | 2.598 |
| | | | 430 mL/min | 64 | 3 | 6.275 | 13.827 | 30.617 | 16.597 | 1.760 |
| 10Pii0297-057 | 50% | Peristaltic | — | — | Initial | 12.023 | 78.103 | 210.546 | 96.559 | 2.542 |
| | | | 430 mL/min | 233 | 1 | 7.076 | 22.109 | 72.290 | 35.202 | 2.950 |
| | | | 500 mL/min | 84 | 2 | 6.552 | 15.662 | 36.631 | 19.635 | 1.921 |

| | | | | | | |
|---|---|---|---|---|---|---|
| Particle Size Stability | Pass 2 after 4 Days | 7.497 | 19.261 | 46.508 | 24.997 | — |

### Example 2

This example demonstrates the stability of a wet-milled, progesterone composition prepared according to an embodiment of the method of the invention. This example also demonstrates that variation of the lecithin content does not affect the particle size distribution of a wet-milled, progesterone composition prepared according to an embodiment of the method of the invention.

A suspension is prepared comprising unmicronized progesterone (D₉₀ 220 μm) (40.0% w/w) and peanut oil (60% w/w) without lecithin. The suspension is pumped into a milling chamber using a peristaltic pump. The suspension is wet-milled for three passes as described in Example 1 with a 50% v/v milling media (bead) load. After the second and third pass, lecithin (0.1% w/w, 0.2% w/w, 0.3% w/w, or 0.4% w/w) is added to the wet-milled progesterone composition.

A sample of each of the second and third pass wet-milled progesterone compositions is collected and subjected to no freeze/thaw cycle or one or more freeze/thaw cycles as set forth in Table 2. In some experiments, the sample is kept at room temperature for two weeks prior to being subject to no freeze/thaw cycle or one or more freeze/thaw cycles, as set forth in Table 2. One freeze/thaw cycle includes 24 hours at each of room temperature (RT), 40° C., and 5° C.

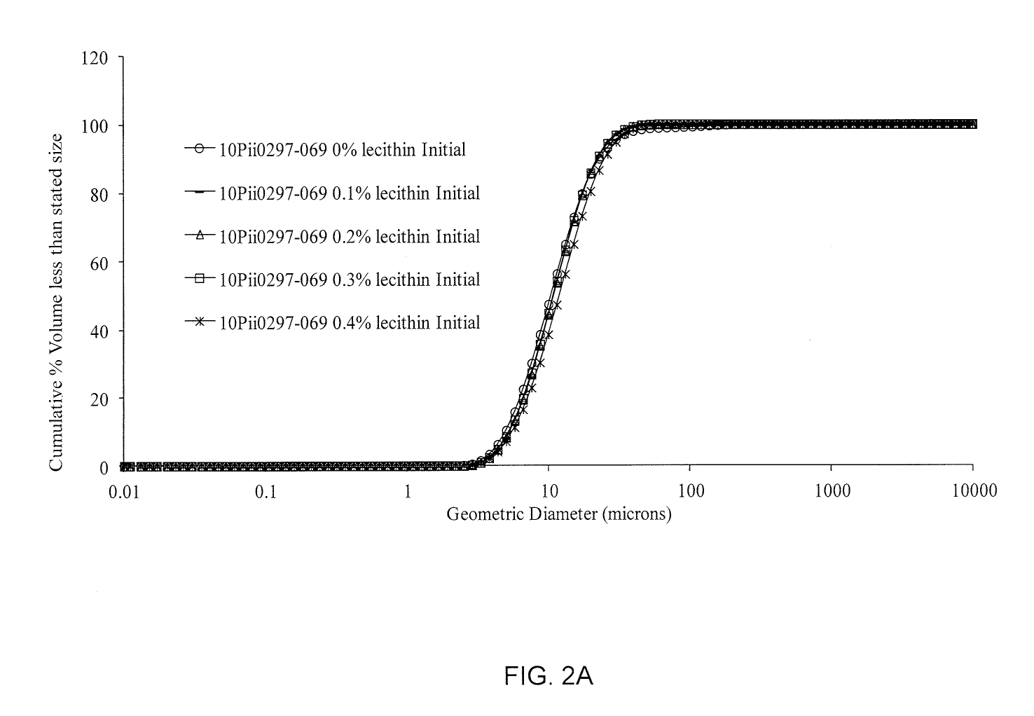

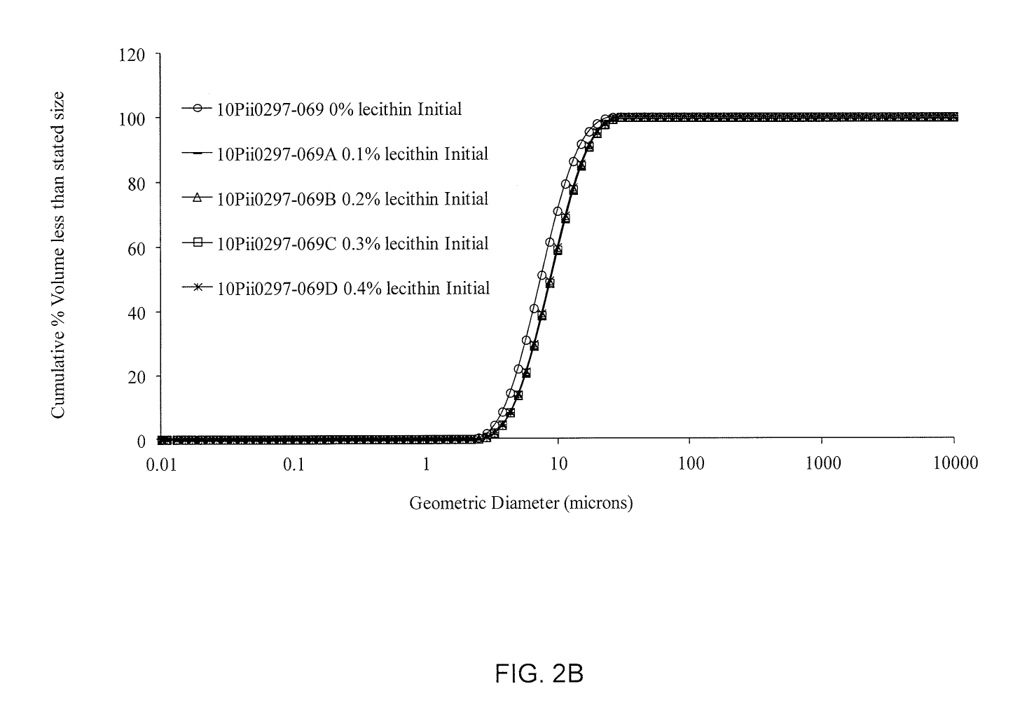

The particle size and viscosity of each sample are measured as described in Example 1. For comparison, the particle size and viscosity of PROMETRIUM (progesterone, USP) capsules (Abbott Laboratories, Abbott Park, Ill.) after one or two freeze/thaw cycles (or an initial sample) are also measured. The results are shown in FIGS. 2A, 2B, 3A, 3B, 4A, 4B, 5A, 5B, 6A, and 6B, Table 2A (second pass), Table 2B (third pass), and Table 2C (PROMETRIUM capsules). “Initial sample” refers to a freshly prepared sample and “freeze thaw” refers to a sample subjected to one or more freeze/thaw cycles as indicated in Table 2A and 2B. A gradual increase in particle size of progesterone particles and a gradual decrease in viscosity of the fill formulation of PROMETRIUM capsules following freeze/thaw cycles are observed (Table 2C).

TABLE 2A

| Sample Type | % Lecithin | d (0.1) | d (0.5) | d (0.9) | D (4, 3) | Span | Viscosity (cP) |
|---|---|---|---|---|---|---|---|
| 2nd Pass - Initial | 0 | 5.706 | 11.953 | 26.314 | 15.455 | 1.724 | 1637.545 |
| | 0.1 | 6.106 | 12.519 | 25.717 | 14.553 | 1.567 | 1753.000 |
| | 0.2 | 6.081 | 12.477 | 25.706 | 14.524 | 1.573 | 1758.727 |
| | 0.3 | 6.050 | 12.417 | 25.798 | 14.546 | 1.590 | 1750.182 |
| | 0.4 | 6.385 | 13.762 | 29.007 | 16.109 | 1.644 | 1766.818 |
| 2nd Pass - Freeze Thaw Cycle 1 | 0.1 | 7.246 | 16.559 | 35.498 | 19.440 | 1.706 | 1625.364 |
| | 0.2 | 7.281 | 16.647 | 35.692 | 19.509 | 1.707 | 1501.182 |
| | 0.3 | 7.383 | 16.713 | 35.651 | 19.594 | 1.691 | 1527.818 |
| | 0.4 | 7.499 | 17.108 | 37.251 | 20.682 | 1.739 | 1467.273 |
| 2nd Pass - Freeze Thaw Cycle 2 | 0.1 | 7.414 | 17.940 | 39.316 | 21.173 | 1.778 | 1561.090 |
| | 0.2 | 7.415 | 17.884 | 39.223 | 21.113 | 1.779 | 1543.640 |
| | 0.3 | 7.414 | 17.632 | 37.911 | 20.635 | 1.730 | 1339.550 |
| | 0.4 | 7.468 | 18.268 | 39.805 | 21.460 | 1.770 | 1267.360 |
| 2nd Pass - Freeze Thaw Cycle 3 | 0.1 | 7.991 | 18.028 | 37.214 | 20.705 | 1.621 | 1912.460 |
| | 0.2 | 7.857 | 17.877 | 37.286 | 20.630 | 1.646 | 1763.820 |
| | 0.3 | 8.210 | 18.517 | 37.467 | 20.973 | 1.580 | 1540.730 |
| | 0.4 | 8.012 | 18.329 | 37.989 | 21.088 | 1.635 | 1535.730 |
| 2nd Pass - Freeze Thaw Cycle 4 | 0.1 | 8.241 | 18.424 | 37.014 | 20.810 | 1.562 | 1812.000 |
| | 0.2 | 8.558 | 19.212 | 38.177 | 21.570 | 1.542 | 1888.545 |
| | 0.3 | 8.801 | 19.751 | 38.981 | 22.102 | 1.528 | 1745.727 |
| | 0.4 | 8.510 | 19.267 | 38.749 | 21.797 | 1.569 | 1617.455 |

TABLE 2B

| Sample Type | % Lecithin | d (0.1) | d (0.5) | d (0.9) | D (4, 3) | Span | Viscosity (cP) |
|---|---|---|---|---|---|---|---|
| 3rd Pass - Initial | 0 | 4.548 | 8.572 | 16.553 | 9.714 | 1.401 | 2071.909 |
| | 0.1 | 5.313 | 10.270 | 19.660 | 11.539 | 1.397 | 2172.182 |
| | 0.2 | 5.264 | 10.131 | 19.414 | 11.398 | 1.397 | 1971.000 |
| | 0.3 | 5.272 | 10.179 | 19.511 | 11.447 | 1.399 | 2117.455 |
| | 0.4 | 5.244 | 10.067 | 19.242 | 11.316 | 1.390 | 2207.545 |
| 3rd Pass - 2 week RT Sample | 0 | 5.352 | 10.422 | 20.049 | 11.726 | 1.410 | 2001.273 |
| | 0.1 | 5.279 | 10.482 | 20.662 | 11.934 | 1.468 | 2047.455 |
| | 0.2 | 5.224 | 10.388 | 20.646 | 11.880 | 1.485 | 1812.182 |
| | 0.3 | — | — | — | — | — | — |
| | 0.4 | 5.188 | 10.274 | 20.381 | 11.372 | 1.479 | 1743.636 |
| 3rd Pass - 2 week RT Sample | 0 | 5.513 | 10.866 | 21.187 | 12.302 | 1.443 | 2582.000 |
| Freeze Thaw Cycle 1 | 0.1 | 5.759 | 11.480 | 22.341 | 12.957 | 1.444 | 1914.727 |
| | 0.2 | 5.767 | 11.571 | 22.530 | 13.027 | 1.449 | 1746.455 |
| | 0.3 | 5.722 | 11.597 | 22.933 | 13.167 | 1.484 | 1869.182 |
| | 0.4 | 5.901 | 11.282 | 21.195 | 12.591 | 1.356 | 1688.727 |
| 3rd Pass - 2 week RT Sample | 0 | 5.897 | 11.204 | 21.065 | 12.534 | 1.354 | 3387.455 |
| Freeze Thaw Cycle 2 | 0.1 | 5.881 | 11.894 | 23.220 | 13.389 | 1.458 | 2183.182 |
| | 0.2 | 6.340 | 12.249 | 22.813 | 13.581 | 1.345 | 1954.364 |
| | 0.3 | 5.979 | 12.146 | 23.571 | 13.628 | 1.448 | 3178.182 |
| | 0.4 | 6.337 | 12.222 | 22.715 | 13.542 | 1.340 | 2105.818 |

TABLE 2C

| | d | d | d | D | | Viscosity |
|---|---|---|---|---|---|---|
| Sample Type | (0.1) | (0.5) | (0.9) | (4, 3) | Span | (cP) |
| Initial | 7.291 | 16.281 | 32.160 | 18.228 | 1.528 | 1694.550 |
| Freeze Thaw | 8.385 | 18.849 | 36.185 | 20.803 | 1.475 | 1525.640 |
| Cycle 1 | | | | | | |
| Freeze Thaw | 8.675 | 19.305 | 36.706 | 21.226 | 1.452 | 1406.550 |
| Cycle 2 | | | | | | |

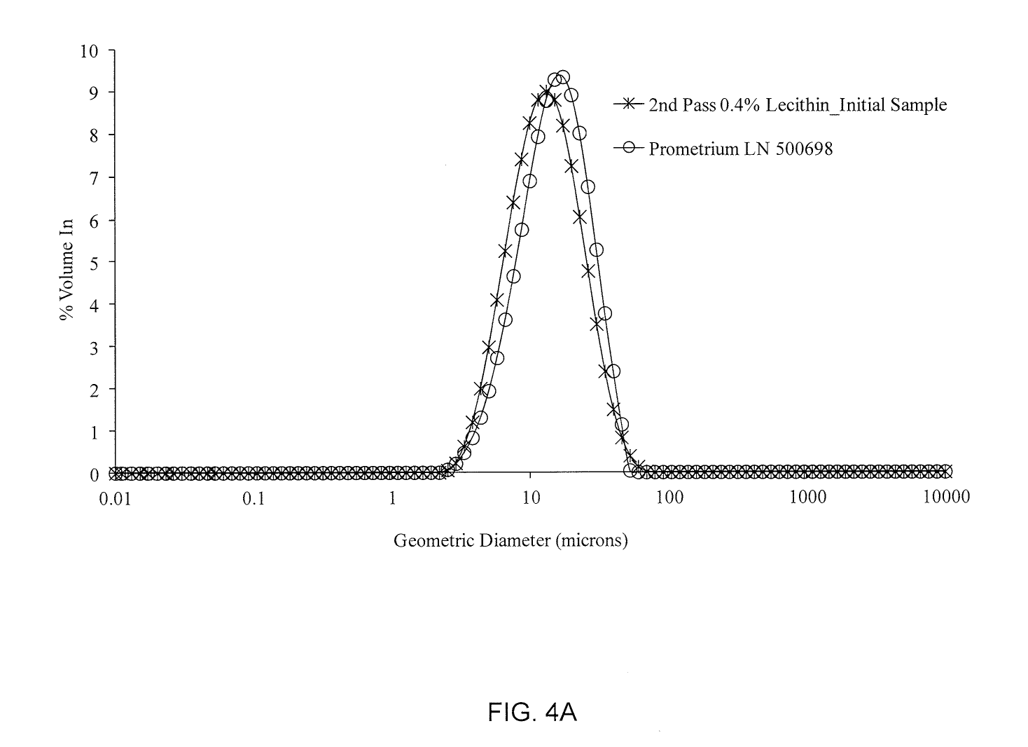

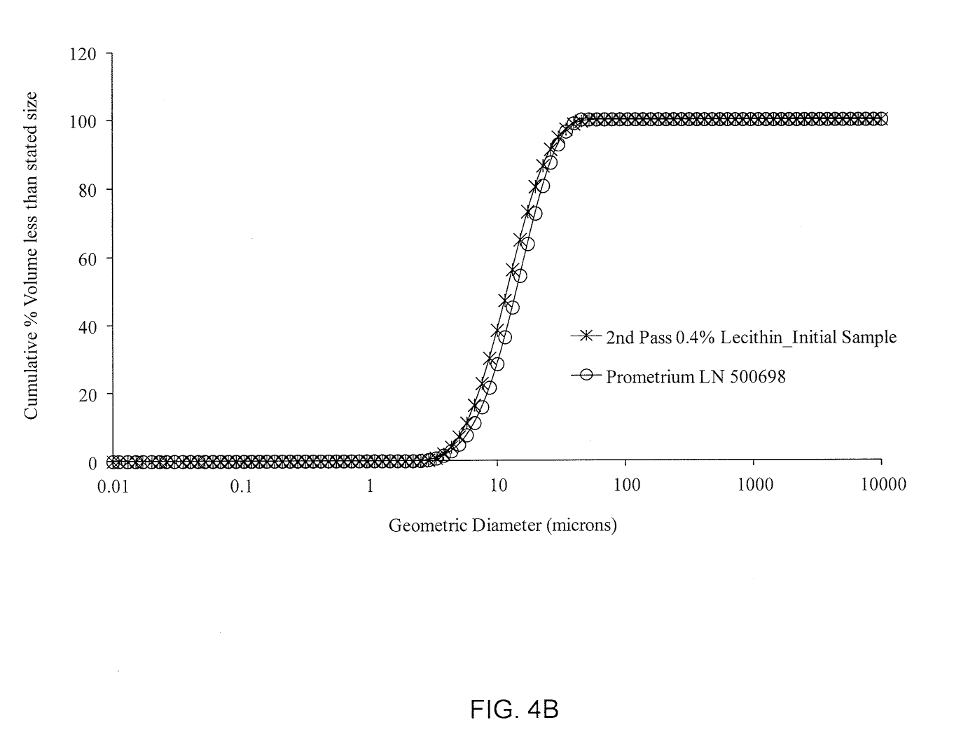

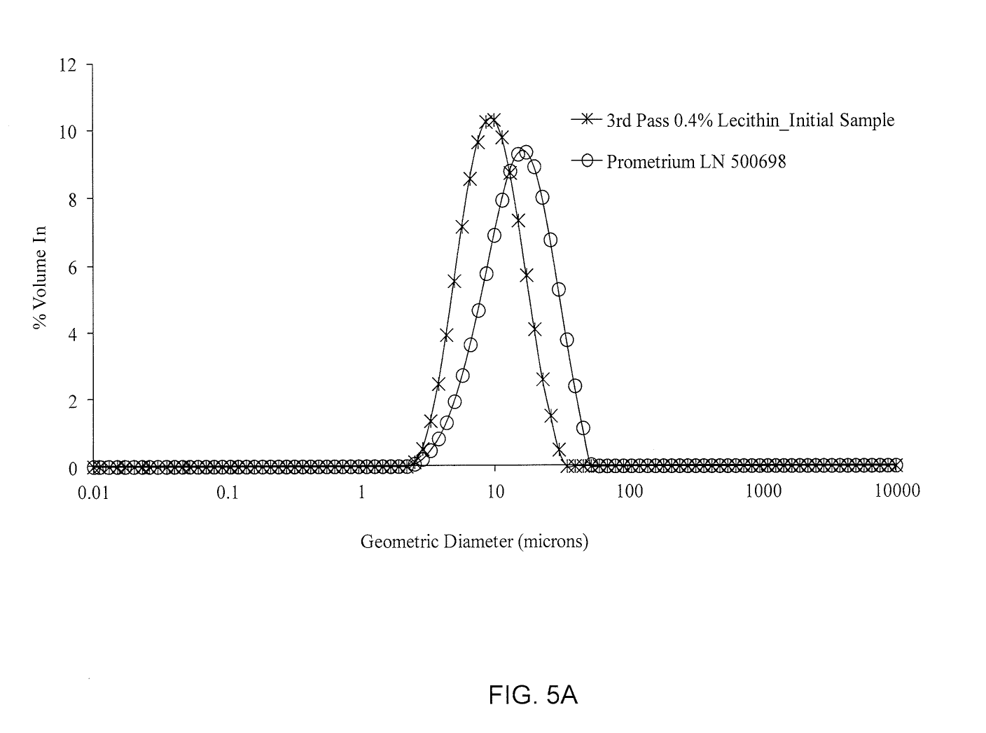

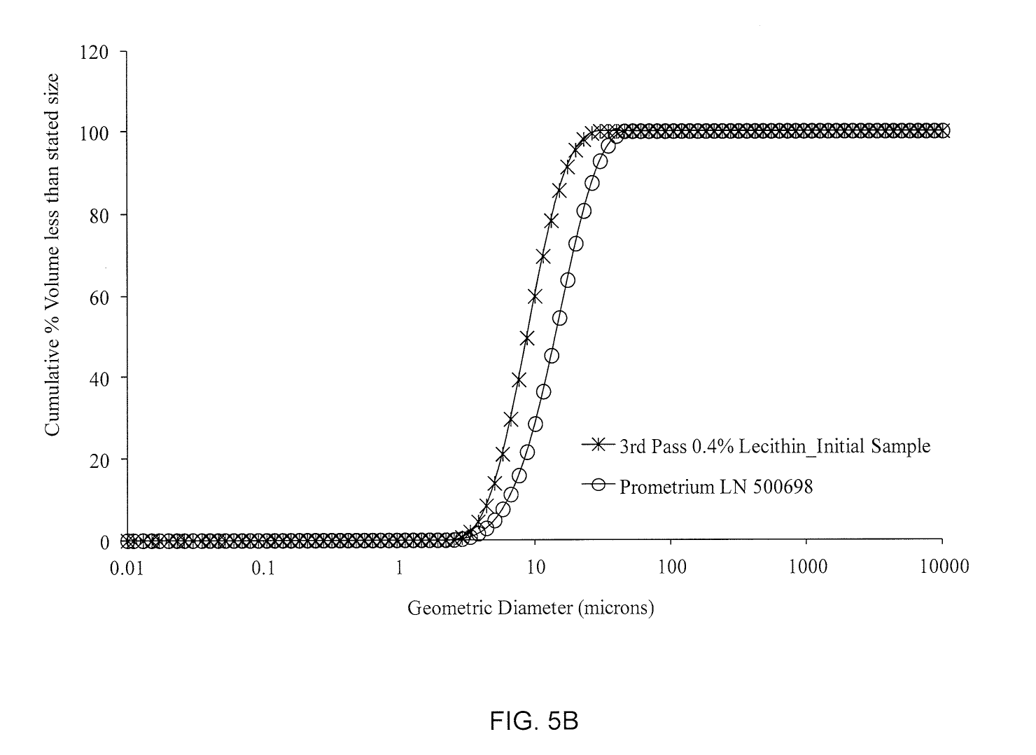

As shown in FIGS. 2A and 2B, variation of the lecithin content between 0% and 0.4% by weight of the mixture does not affect the particle size distribution of a wet-milled progesterone composition prepared according to an embodiment of the inventive method. As shown in FIGS. 4A, 4B, 5A, and 5B, the particle size distribution of a wet-milled progesterone composition having 0.4% lecithin and which was prepared according to an embodiment of the inventive method is comparable to that of PROMETRIUM capsules.

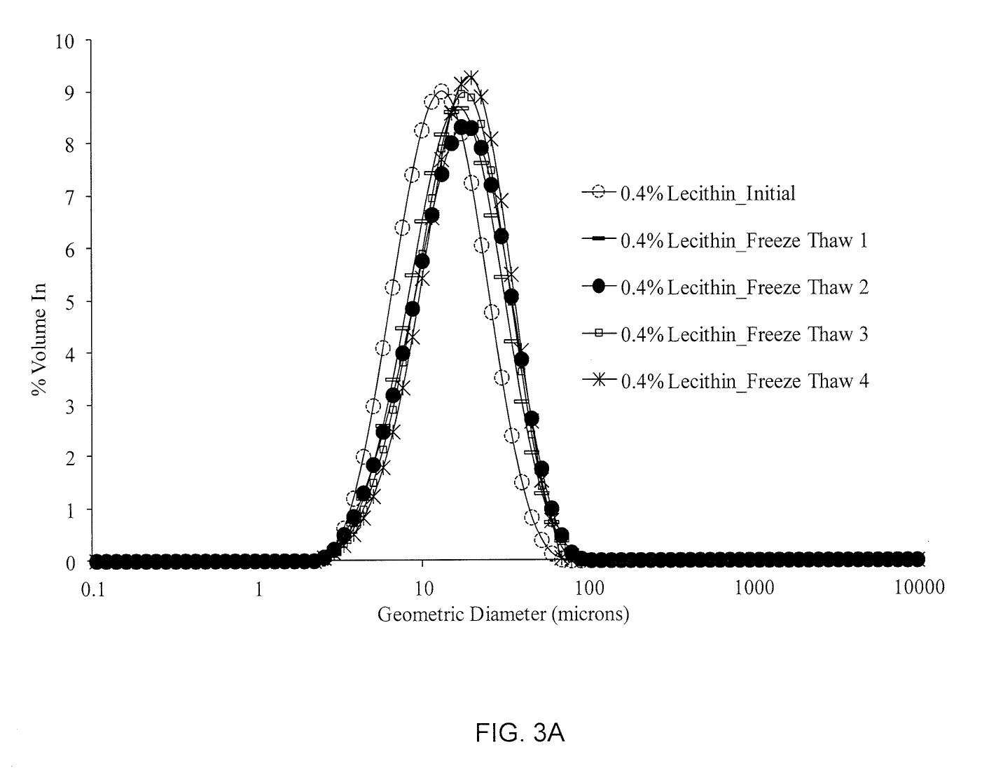

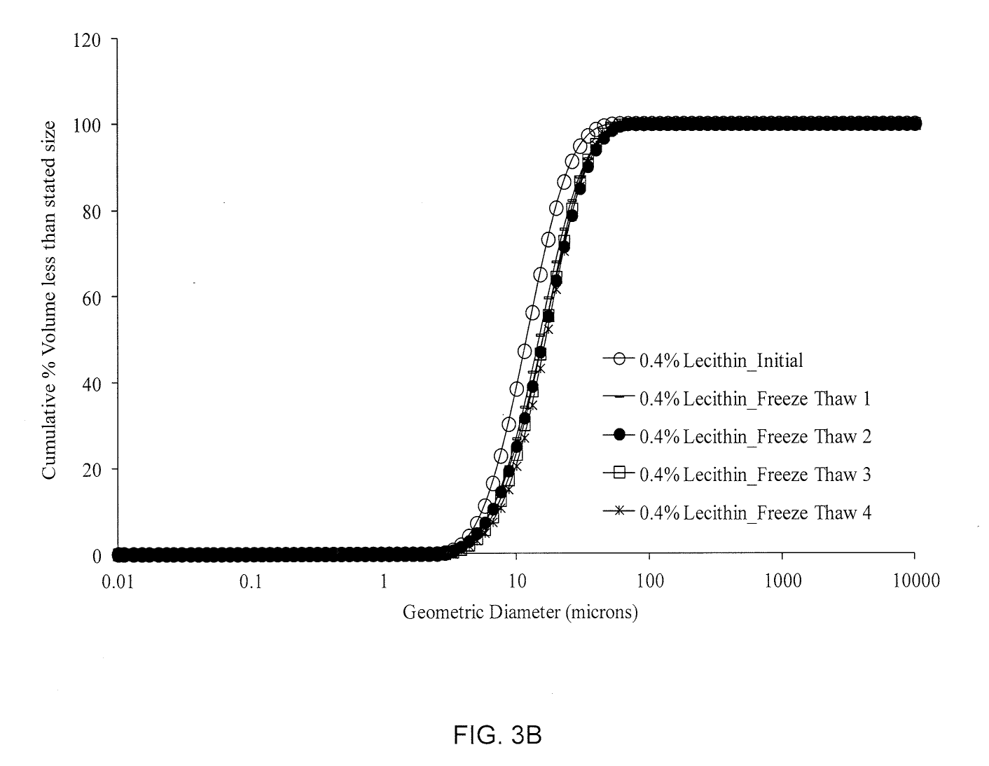

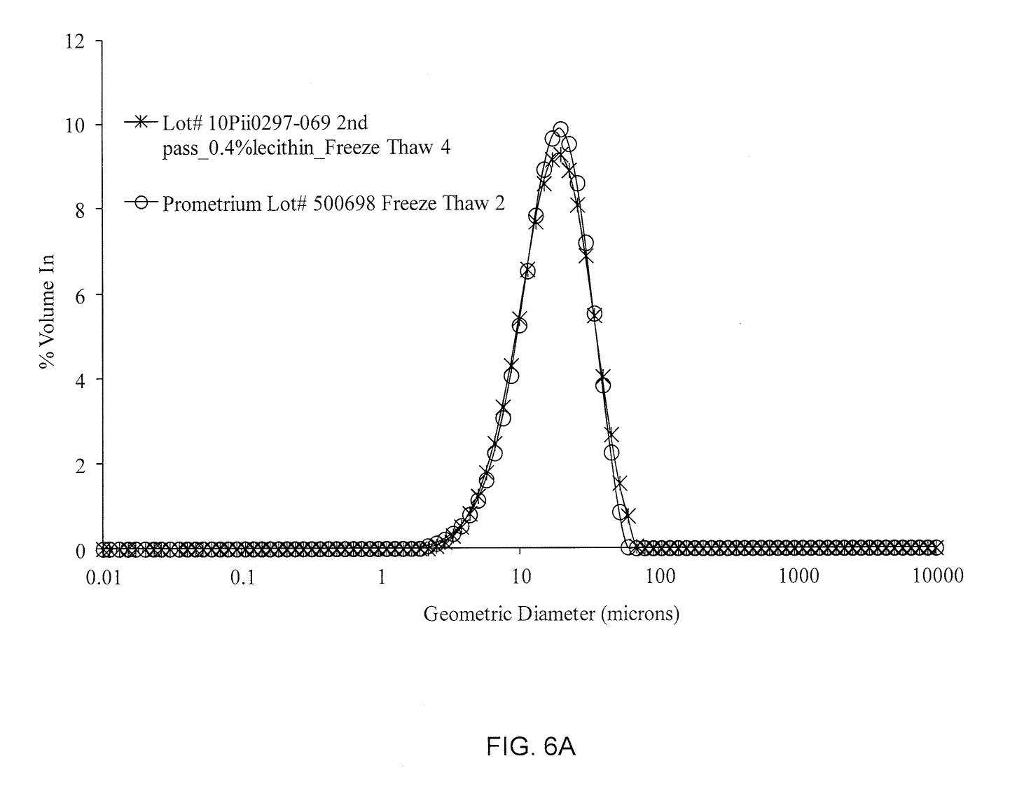

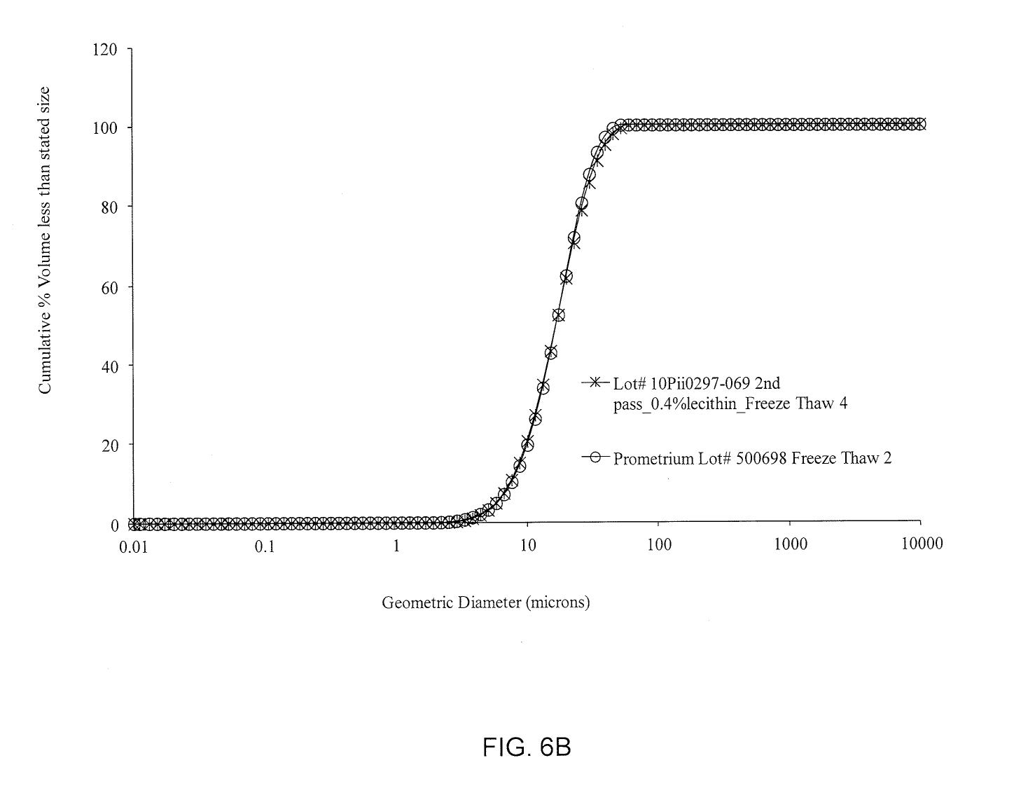

As shown in FIGS. 3A and 3B, the particle size distribution of a wet-milled progesterone composition prepared according to an embodiment of the inventive method is stable after four freeze/thaw cycles. As shown in FIGS. 6A and 6B, the stability of the particle size distribution of a wet-milled progesterone composition prepared according to an embodiment of the inventive method is comparable to that of PROMETRIUM capsules after four freeze/thaw cycles.

### Example 3

This example demonstrates the viscosity and particle size of a wet-milled, progesterone composition prepared according to a scaled-up embodiment of the method of the invention. This example also demonstrates the influence of the percentage of media milling (beads) on particle size distribution.

A suspension is prepared comprising unmicronized progesterone (D₉₀ 220 μm) (40.0% w/w) and peanut oil (60% w/w) without lecithin. The suspension is pumped into a milling chamber using a positive displacement pump (PDP). The suspension is wet-milled for three passes as described in Example 1 using 65% v/v milling media (bead) load in a 1.4 L milling chamber. The particle size and viscosity of each sample are measured as described in Example 1. The results are shown in Table 3A.

In a separate experiment, a suspension is prepared and wet-milled according to the same procedure except that the percentage of milling media (bead) load is varied as set forth in Table 3B. The particle size of each sample is measured as described in Example 1. The results are shown in Table 3B.

TABLE 3A

| Lot # | % Beads | Pump Type | Input | Output | Pass# | D10 | D50 | D90 | D (4, 3) | Span | Viscosity (cP) |
|---|---|---|---|---|---|---|---|---|---|---|---|
| 11Pii0192-040 | 65.00% | PDP | 275 g/min | 275 | 2 | 8.521 | 16.449 | 29.681 | 17.938 | 1.286 | 1992.34 |
| | | | | 275 | 3 | 7.566 | 13.835 | 24.258 | 15.026 | 1.207 | 2484.47 |

TABLE 3B

| Lot # | % Beads | Pump Type | Input | Output | Pass# | D10 | D50 | D90 | D (4, 3) | Span |
|---|---|---|---|---|---|---|---|---|---|---|
| 11Pii0052-052 | 53.57% | Peristaltic | — | — | Initial | 22.929 | 101.240 | 242.947 | 119.103 | 2.173 |
| | | | | | 1 | 10.882 | 21.678 | 40.566 | 23.945 | 1.369 |
| | | | | | 2 | 7.350 | 13.645 | 24.135 | 14.843 | 1.23 |
| 11Pii0052-052C | 53.57% | Peristaltic | — | — | Initial | 22.929 | 101.240 | 242.947 | 119.103 | 2.173 |
| | | | | | 1 | 8.123 | 19.621 | 45.217 | 25.084 | 1.891 |
| | | | | | 2 | 20.004 | 36.838 | 66.759 | 41.373 | 1.269 |
| | | | | | 3 | 15.138 | 33.365 | 68.763 | 40.365 | 1.607 |
| | | | | | 5 | 16.788 | 31.837 | 56.526 | 34.503 | 1.248 |
| 11Pii0052-052E | 53.57% | Peristaltic | — | — | Initial | 22.929 | 101.240 | 242.947 | 119.103 | 2.173 |
| | | | | 901 | 1 | 30.629 | 68.510 | 148.840 | 80.358 | 1.725 |
| | | | | 857 | 2 | 21.542 | 47.958 | 98.504 | 54.676 | 1.605 |
| | | | | 800 | 3 | 18.452 | 39.373 | 77.638 | 44.201 | 1.503 |
| 11Pii0052-052G | 53.57% | Peristaltic | — | — | Initial | 22.929 | 101.240 | 242.947 | 119.103 | 2.173 |
| | | | | | 3 | 7.195 | 16.753 | 36.333 | 19.753 | 1.739 |
| | | | | | 4 | 6.253 | 12.934 | 26.625 | 14.658 | 1.498 |
| 11Pii0052-052H | 50% | Peristaltic | 430 mL/min | — | Initial | 22.929 | 101.240 | 242.947 | 119.103 | 2.173 |
| | | | | 187 | 1 | 9.115 | 28.758 | 87.678 | 41.783 | 2.732 |
| | | | | 100 | 2 | 6.255 | 14.152 | 31.508 | 16.946 | 1.784 |
| | | | | 35 | 3 | 5.398 | 10.516 | 20.519 | 11.936 | 1.438 |
| 11Pii0052-052I | 65% | Peristaltic | — | — | Initial | 22.929 | 101.240 | 242.947 | 119.103 | 2.173 |
| | | | | 530 | 1 | 16.697 | 35.061 | 73.427 | 45.464 | 1.618 |
| | | | | 250 | 2 | 10.911 | 22.914 | 43.543 | 25.305 | 1.424 |
| | | | | 45 | 3 | 8.352 | 16.594 | 30.860 | 18.297 | 1.356 |
| 11Pii0052-052J | 65% | Peristaltic | — | — | Initial | 22.929 | 101.240 | 242.947 | 119.103 | 2.173 |
| | | | | 278 | 1 | 14.518 | 34.272 | 69.552 | 38.702 | 1.606 |
| | | | | 239 | 2 | 12.095 | 24.432 | 45.851 | 26.969 | 1.382 |
| | | | | 265 | 2 | 10.423 | 20.880 | 38.688 | 22.944 | 1.354 |
| 11Pii0052-052K | 50% | Peristaltic | — | — | Initial | 79.035 | 163.879 | 297.117 | 176.538 | 1.331 |
| | | | | 212 | 3 | 17.321 | 32.257 | 56.381 | 34.741 | 1.211 |
| | | | | 175 | 4 | 13.958 | 27.752 | 50.457 | 30.147 | 1.315 |

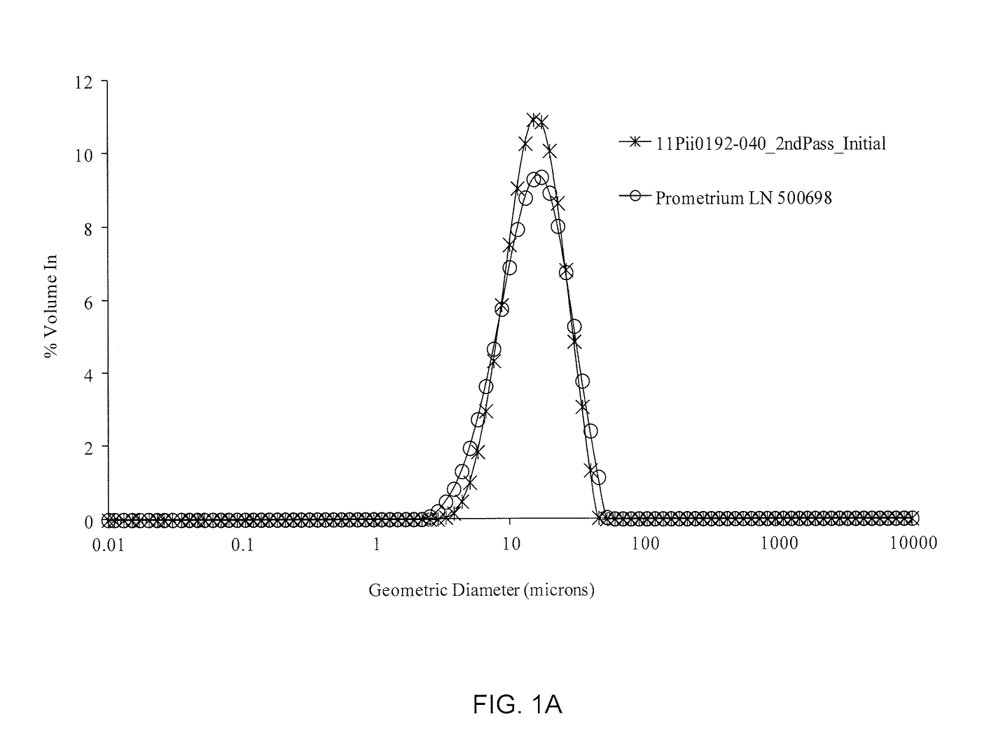

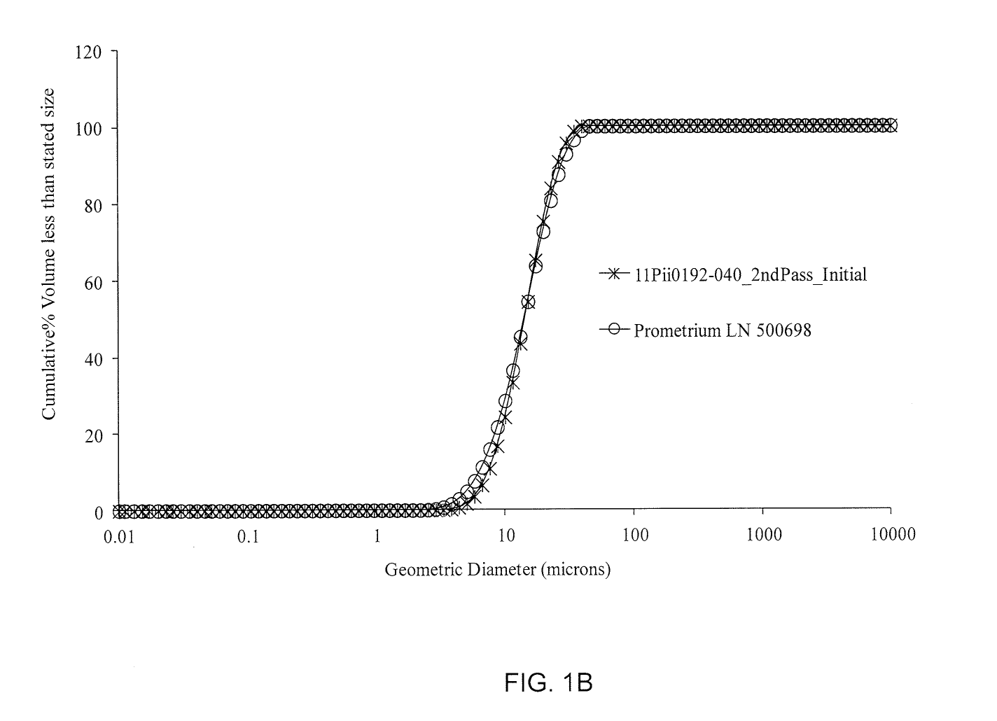

The particle size distribution of the second pass sample of lot 11Pii0192-040 of Table 3 (0% lecithin) is compared with that of PROMETRIUM capsules. The percent volume having a given geometric diameter between 0.01 micron and 1,000 microns is shown in FIG. 1A. FIG. 1B shows the cumulative percent volume that is less than a given geometric diameter between 0.01 micron and 1,000 microns. As shown in FIGS. 1A and 1B, particle size distribution of a wet-milled, progesterone composition prepared according to an embodiment of the inventive method is comparable to that of PROMETRIUM capsules.

### Example 4

This example demonstrates the particle size of a wet-milled, progesterone composition prepared according to an embodiment of the method of the invention that has been milled for various time periods.

A suspension is prepared comprising unmicronized progesterone (40.0% w/w) (D₉₀ 220 μm), soybean/sesame oil (59.8% w/w), and soy lecithin (0.2% w/w). The suspension is pumped into a milling chamber using a peristaltic pump. The suspension is wet-milled as described in Example 1 for the various durations set forth in Table 4 with a 50% v/v milling media (bead) load using static milling. The coarse suspension is transferred into the mill and milling continues. A sample is collected at the time points set forth in Table 4. The particle size of each sample is measured as described in Example 1. The results are shown in Table 4. As shown in Table 4, in general, longer durations of wet-milling provide smaller particle sizes.

TABLE 4

| Lot # | % Beads | Pump Type | Input | Output | Milling Time | D₁₀ | D₅₀ | D₉₀ | D_{(4, 3)} | Span |
|---|---|---|---|---|---|---|---|---|---|---|
| 10Pii0080-043 | | Peristaltic | | | Initial | 18.118 | 92.444 | 220.844 | 107.734 | 2.193 |
| | | | | | 2 min | 8.205 | 20.486 | 49.255 | 27.780 | 2.004 |
| | | | | | 4 min | 6.021 | 12.932 | 26.791 | 14.906 | 1.606 |
| 10Pii0080-048 | 50% | Peristaltic | 430 mL/min | — | Initial | 13.08 | 82.493 | 219.581 | 101.186 | 2.503 |
| | | | | — | 2 min | 7.227 | 18.769 | 48.218 | 24.957 | 2.184 |
| | | | | — | 4 min | 6.557 | 15.109 | 34.594 | 18.881 | 1.856 |
| | | | | — | 6 min | 6.346 | 13.949 | 30.294 | 16.571 | 1.717 |
| | | | | | 8 min | 5.696 | 11.668 | 24.154 | 13.592 | 1.582 |
| 10Pii0080-050 | 50% | Peristaltic | 430 mL/min | — | 5 min | 6.507 | 14.262 | 31.913 | 17.032 | 1.781 |
| | | | | — | 6 min | 6.55 | 15.513 | 36.007 | 18.798 | 1.899 |
| 10Pii0080-053 | 50% | Peristaltic | 430 mL/min | — | Initial | 13.493 | 79.149 | 211.829 | 97.838 | 2.506 |
| | | | | | 9 min | 7.23 | 17.424 | 43.096 | 24.467 | 2.058 |
| | | | | | 10 min | 6.751 | 15.724 | 43.833 | 22.408 | 2.358 |
| | | | | | 12 min | 6.156 | 14.735 | 49.014 | 25.212 | 2.908 |
| 10Pii0080-059 | 50% | Peristaltic | 430 mL/min | | 10 min | 5.721 | 13.899 | 47.375 | 25.055 | 2.997 |
| 10Pii0080-067 | 50% | Peristaltic | 430 mL/min | | Initial | 16.806 | 88.542 | 225.323 | 106.934 | 2.355 |
| | | | | | 5 min | 7.717 | 20.169 | 54.347 | 28.532 | 2.312 |

### Example 5

This example demonstrates the particle size of a wet-milled, progesterone composition comprising mixed oils prepared according to an embodiment of the method of the invention.

A suspension is prepared and wet-milled according to the procedure of Example 4 except that the suspension comprises unmicronized progesterone (D₉₀ 220 μm) (40.0% w/w), a combination of soybean oil and sesame oil (59.6% w/w), and soy lecithin (0.4% w/w), the percentage of milling media (bead) load and the number of passes are varied as set forth in Table 5. The particle size of each sample is measured as described in Example 1. The results are shown in Table 5.

TABLE 5

| Lot # | % Beads | Pass # | D10 | D50 | D90 | D (4, 3) | Span |
|---|---|---|---|---|---|---|---|
| 11Pii0052-016 | 57.50% | Initial | 16.445 | 85.579 | 214.884 | 102.650 | 2.319 |
| | | 1 | 7.230 | 20.139 | 59.967 | 29.786 | 2.619 |
| 11Pii0052-020 | 65% | Initial | 16.445 | 85.579 | 214.884 | 102.650 | 2.319 |
| | | 1 | 7.083 | 20.605 | 57.573 | 28.906 | 2.450 |
| | | 2 | 6.887 | 17.778 | 42.666 | 21.909 | 2.013 |
| 11Pii0052-024 | 70% | Initial | 16.445 | 85.579 | 214.884 | 102.650 | 2.319 |
| | | 1 | 7.331 | 20.634 | 57.808 | 29.456 | 2.446 |
| 11Pii0052-028 | 65% | Initial | 13.808 | 89.967 | 221.198 | 105.354 | 2.305 |
| | | 1 | 6.922 | 19.008 | 57.781 | 29.486 | 2.676 |
| | | 2 | 5.433 | 12.823 | 31.894 | 16.322 | 2.064 |
| | | 3 | 4.956 | 9.865 | 20.064 | 11.422 | 1.532 |
| 10Pii0080-070 | 60% | 1 | 5.703 | 12.314 | 26.919 | 14.701 | 1.723 |
| 10Pii0080-073 | 50% | Initial | 15.31 | 88.828 | 218.607 | 104.515 | 2.289 |
| | | 1 | 8.155 | 22.735 | 61.611 | 30.884 | 2.351 |

### Example 6

This example demonstrates encapsulation of a wet-milled progesterone composition prepared according to an embodiment of the method of the invention. This example also demonstrates the particle size distribution before and after the addition of lecithin and before and after the encapsulation process.

Samples of the wet-milled progesterone composition prepared according to Example 3 are collected after the first and second passes and 0.4% lecithin is added. The addition of lecithin includes mixing the lecithin into the samples. To evaluate whether the lecithin, and the further mixing, have any effect on the particle size distribution of the samples, the particle size distribution is measured before and after the addition of lecithin as described in Example 1 and as set forth in Table 7 and compared to that of PROMETRIUM capsules. The results are shown in Table 7. As shown in Table 7, the addition of lecithin, including the mixing, does not significantly alter the particle size distribution.

The resulting mixture is encapsulated in a gel comprising gelatin 150 (bloom limed bone), glycerin, purified water, opatint white, and FD&C Yellow No. 6. The composition of the fill material for encapsulation is set forth in Table 6. The encapsulation process includes further mixing of the suspension that will form the fill material. To evaluate whether the further mixing of the encapsulation process has any effect on the particle size distribution of the fill material, the particle size distribution of the fill material is measured before and after encapsulation as described in Example 1 and as set forth in Table 7 and compared to that of PROMETRIUM capsules. The results are shown in Table 7. As shown in Table 7, the encapsulation process that including the further mixing does not significantly alter the particle size distribution.

TABLE 6

Composition
Formulation, mg

| | Formulation C | Formulation D |
|---|---|---|
| Progesterone | 200 | 200 |
| Peanut oil | 318 | 348 |
| Lecithin | 2 | 2 |
| Fill Weight, mg | 520 | 550 |
| Material Used | From 1^{st} Pass | From 2^{nd} Pass |

TABLE 7

| | Particle Size Distribution, microns | | | | | Viscosity |
|---|---|---|---|---|---|---|
| Sample | D10 | D50 | D90 | D (4, 3) | Span | (cP) |

1^{st} Pass - Before Encapsulation

| | | | | | | |
|---|---|---|---|---|---|---|
| 1^{st} Pass - Before Lecithin Addition | 8.008 | 18.408 | 41.032 | 23.578 | 1.794 | — |
| 1^{st} Pass with Lecithin | 8.707 | 20.469 | 46.338 | 27.389 | 1.838 | 1040 |
| Fill Wt. 520 mg (Formulation C) | | | | | | |

1^{st} Pass - After Encapsulation

| | | | | | | |
|---|---|---|---|---|---|---|
| 1^{st} Pass with Lecithin | 9.129 | 20.924 | 45.789 | 26.796 | 1.752 | — |
| Fill Wt. 520 mg (Formulation C) | | | | | | |

2^{nd} Pass - Before Encapsulation

| | | | | | | |
|---|---|---|---|---|---|---|
| 2^{nd} Pass - Before Lecithin addition | 6.600 | 13.289 | 25.324 | 14.796 | 1.409 | — |
| 2^{nd} Pass with Lecithin | 6.556 | 14.202 | 29.098 | 16.296 | 1.587 | 945 |
| Fill Wt. 550 mg (Formulation D) | | | | | | |
| 1^{st} Pass mixed with Lecithin and then | 6.255 | 12.761 | 24.879 | 14.373 | 1.459 | 1381 |
| Milled to 2^{nd} Pass | | | | | | |
| Fill Wt. 550 mg (Formulation D) | | | | | | |

2^{nd} Pass - After Encapsulation

| | | | | | | |
|---|---|---|---|---|---|---|
| 2^{nd} Pass with Lecithin | 7.316 | 15.378 | 29.805 | 17.177 | 1.462 | — |
| Fill Wt. 550 mg (Formulation D) | | | | | | |
| PROMETRIUM 200 mg Lot 500698 | 7.291 | 16.281 | 32.160 | 18.228 | 1.528 | 1694 |

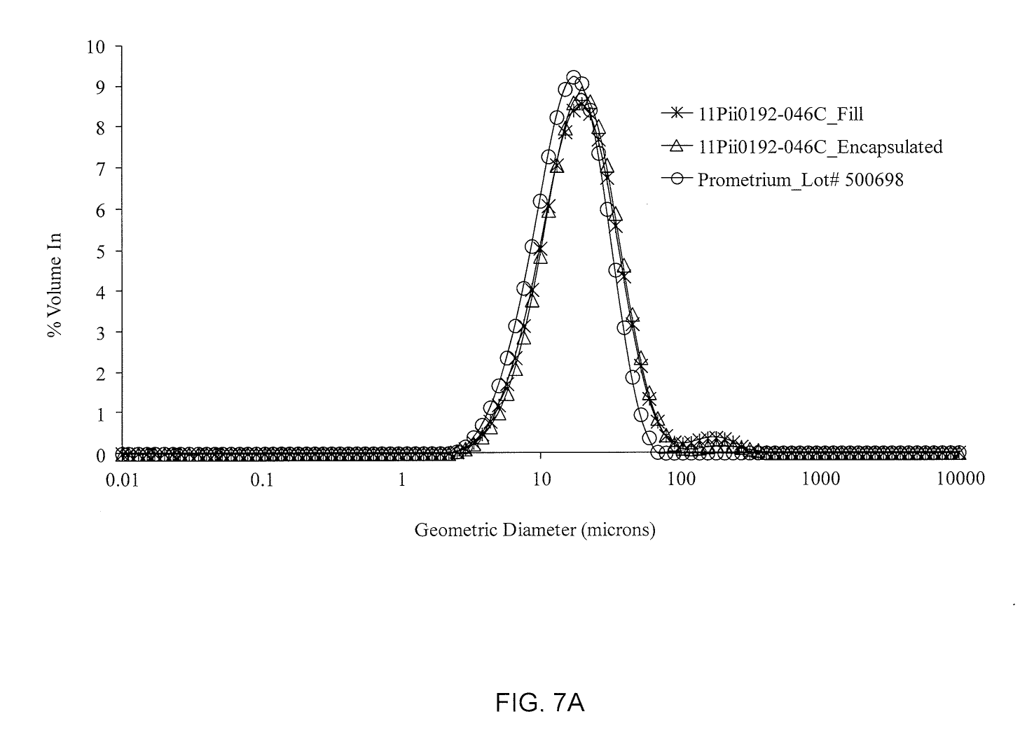

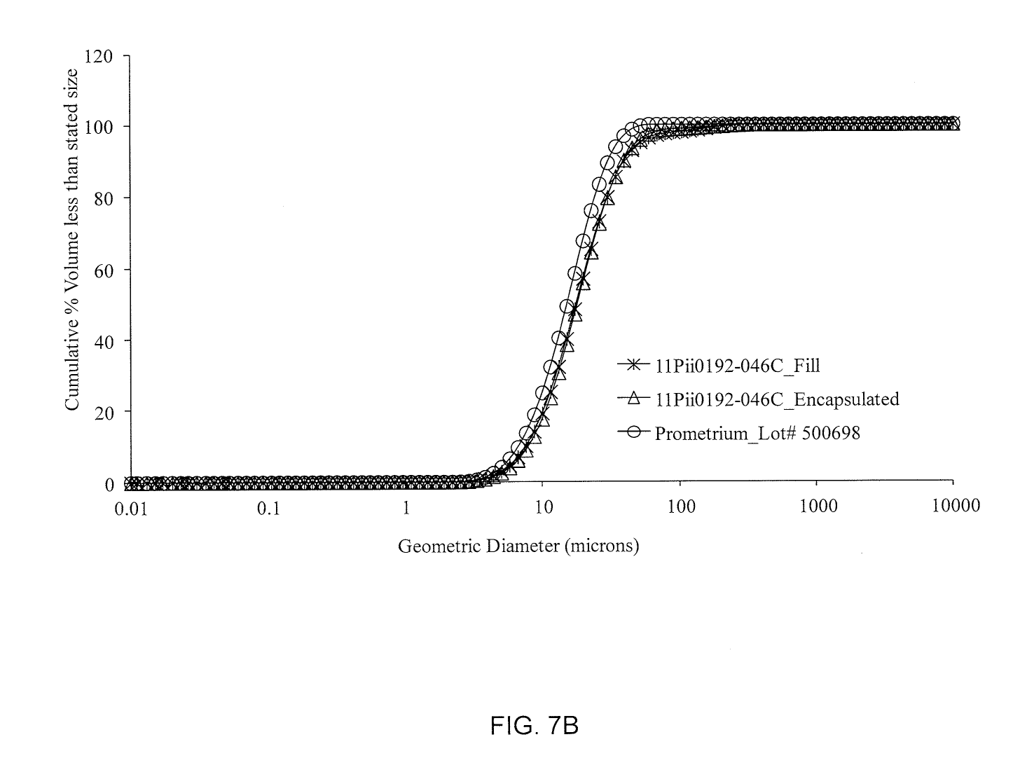

The particle size distributions of the fill material after encapsulation of the first pass and second pass materials are compared with that of PROMETRIUM capsules. The percent volume having a given geometric diameter between 0.01 micron and 1,000 microns is shown in FIG. 7A. FIG. 7B shows the cumulative percent volume that is less than a given geometric diameter between 0.01 micron and 1,000 microns. As shown in FIGS. 7A and 7B (Formulation C of Table 6), the particle size distribution of a wet-milled, progesterone composition prepared according to an embodiment of the inventive method (fill material) is comparable to that of PROMETRIUM capsules before and after encapsulation.

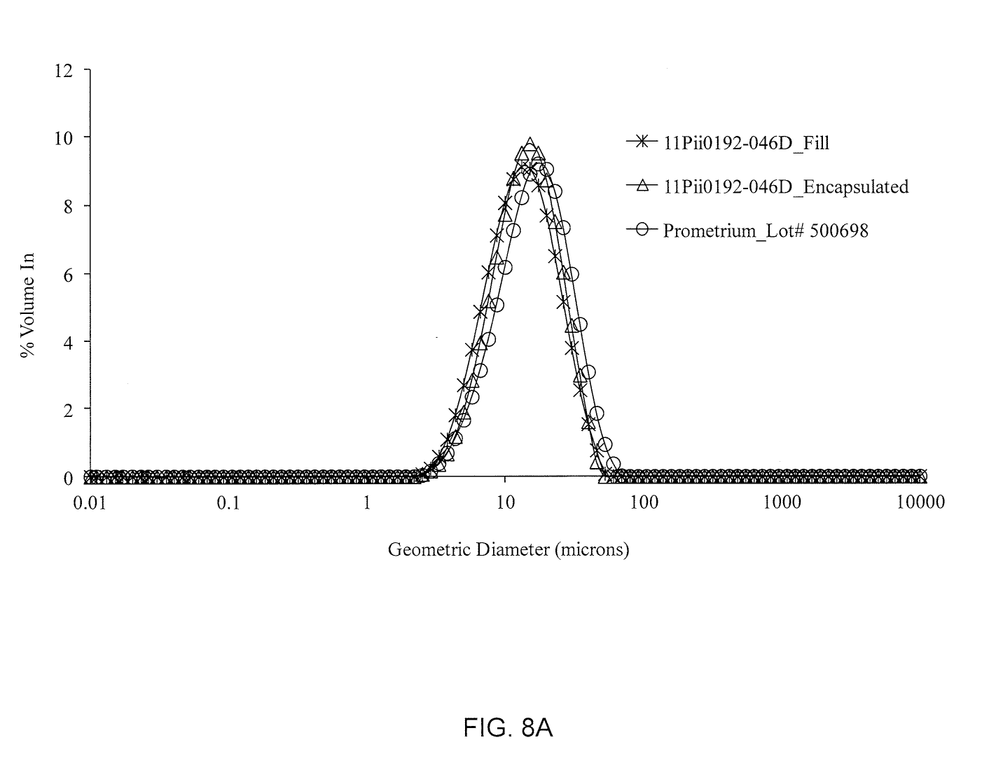

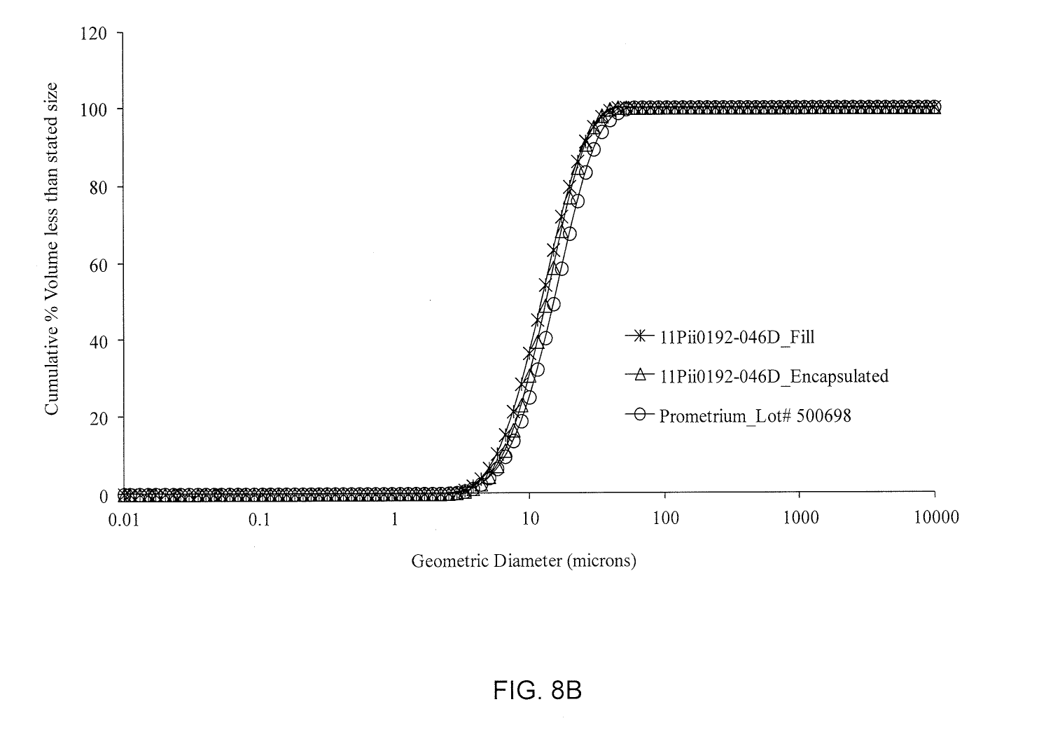

The particle size distribution of the second pass sample of lot 11Pii0192-046 of Table 7 (with lecithin) before and after encapsulation is compared with that of PROMETRIUM capsules. The percent volume having a given geometric diameter between 0.01 micron and 1,000 microns is shown in FIG. 8A. FIG. 8B shows the cumulative percent volume that is less than a given geometric diameter between 0.01 micron and 1,000 microns. As shown in FIGS. 8A and 8B (Formulation D of Table 6), the particle size distribution of a wet-milled, progesterone composition prepared according to an embodiment of the inventive method (fill material) is comparable to that of PROMETRIUM capsules before and after encapsulation.

### Example 7

This example demonstrates the particle size of a wet-milled, progesterone composition prepared according to a scaled-up embodiment of the method of the invention.

A suspension is prepared comprising unmicronized progesterone (D₉₀ 220 μm) (15 kg) and peanut oil (22.35 kg). The suspension is pumped into a milling chamber using a positive displacement pump (PDP). The suspension is wet-milled for one pass as described in Example 1 using 65% v/v milling media (bead) load in a 1.4 L milling chamber to produce fill material. Lecithin (150 g) is added to the milled suspension to yield 37.5 kg milled suspension for encapsulation. The particle size of the fill material for each of 100 mg and 200 mg progesterone fill material is measured as described in Example 1 before encapsulation and compared to that of PROMETRIUM capsules. The results are shown in Table 8.

TABLE 8

| | | | D90, |
|---|---|---|---|
| Sample | D 10, microns | D 50, microns | microns |
| Prometrium RLD Lot 500958 | 7.629 | 16.518 | 31.457 |
| Prometrium RLD Lot 500958 | 7.673 | 16.431 | 31.005 |
| Pii - 100 mg Strength | 7.068 | 15.817 | 35.114 |
| Pii - 200 mg Strength Sublot A | 6.384 | 13.943 | 31.546 |
| Pii - 200 mg Strength Sublot B | 5.825 | 13.081 | 32.394 |

As shown in Table 8, the particle size distribution of a scaled-up, wet-milled, progesterone composition prepared according to an embodiment of the inventive method is comparable to that of PROMETRIUM capsules.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method of preparing a pharmaceutical composition comprising:
(a) combining progesterone particles with a liquid carrier to provide a mixture;
(b) wet-milling the mixture to provide a wet-milled progesterone composition; and
(c) processing the wet-milled progesterone composition to provide a pharmaceutical composition.

2. A method of preparing a pharmaceutical composition comprising wet-milling progesterone particles in a liquid carrier to provide a wet-milled progesterone composition and processing the wet-milled progesterone composition to provide a pharmaceutical composition.

3. The method of claim 1, wherein the pharmaceutical composition is an oral pharmaceutical composition.

4. The method of claim 1, wherein the pharmaceutical composition is a capsule.

5. The method of claim 1, wherein the pharmaceutical composition is a soft-shelled capsule.

6. The method of claim 1, wherein the pharmaceutical composition is a hard-shelled capsule.

7. The method of claim 1, wherein processing the wet-milled progesterone composition comprises encapsulating the wet-milled progesterone composition to provide a capsule.

8. The method of claim 1, wherein wet-milling provides progesterone particles having a cumulative size distribution D₉₀ from about 10 μm to about 70 μm.

9. The method of claim 1, wherein wet-milling provides progesterone particles having a cumulative size distribution D₅₀ from about 5 μm to about 30 μm.

10. The method of claim 1, wherein wet-milling provides progesterone particles having a cumulative size distribution D₁₀ from about 2 μm to about 15 μm.

11. The method of claim 1, wherein the liquid carrier comprises any one or more of a long-chain glyceride, a free fatty acid, a fatty acid ester, and a medium-chain glyceride.

12. The method of claim 11, wherein the liquid carrier comprises at least one long-chain glyceride, and the at least one long-chain glyceride is selected from the group consisting of any one or more of peanut oil, soybean oil, sunflower oil, olive oil, sesame oil, colza oil, almond oil, safflower oil, corn oil, linseed oil, rapeseed oil, evening primrose oil, grape seed oil, cottonseed oil, flaxseed oil, and menhaden oil.

13. The method of claim 11, wherein the liquid carrier comprises at least one fatty acid ester, and the at least one fatty acid ester is selected from the group consisting of glycerol mono-, di, or tri-esters of a long or medium chain fatty acid.

14. The method of claim 11, wherein the liquid carrier comprises at least one fatty acid ester, and the at least one fatty acid ester is selected from the group consisting of propylene glycol mono- and di-esters of a long or medium chain fatty acid.

15. The method of claim 1, wherein the liquid carrier comprises at least one phospholipid.

16. The method of claim 15, wherein the phospholipid is lecithin.

17. The method of claim 1, wherein the liquid carrier comprises at least one lipophilic surfactant.

18. The method of claim 1, wherein the mixture comprises from about 4% to about 80% progesterone by weight of the mixture.

19. The method of claim 1, wherein the mixture comprises from about 10% to about 70% progesterone by weight of the mixture.

20. The method of claim 1, wherein the mixture comprises from about 20% to about 60% progesterone by weight of the mixture.

21. The method of claim 1, wherein the mixture comprises from about 40% to about 50% progesterone by weight of the mixture.

22. The method of claim 1, wherein the mixture comprises from about 20% to about 96% liquid carrier by weight of the mixture.

23. The method of claim 1, wherein the mixture comprises from, about 30% to about 90% liquid carrier by weight of the mixture.

24. The method of claim 1, wherein the mixture comprises from about 40% to about 80% liquid carrier by weight of the mixture.

25. The method of claim 1, wherein the mixture comprises from about 50% to about 60% liquid carrier by weight of the mixture.

26. The method of claim 1, wherein the mixture comprises from about 0.05% to about 5% phospholipid and/or lipophilic surfactant by weight of the mixture.

27. A pharmaceutical composition prepared according to the method of claim 1.

28. A method of preparing a wet-milled progesterone composition comprising:
(a) combining progesterone particles with a liquid carrier, optionally with at least one phospholipid and/or at least one lipophilic surfactant, to provide a mixture; and
(b) wet-milling the mixture to provide a wet-milled progesterone composition.

29. The method of claim 28, wherein wet-milling provides progesterone particles having a cumulative size distribution D₉₀ from about 10 μm to about 70 μm.

30. The method of claim 28, wherein wet-milling provides progesterone particles having a cumulative size distribution D₅₀ from about 5 μm to about 30 μm.

31. The method of claim 28, wherein wet-milling provides progesterone particles having a cumulative size distribution D₁₀ from about 2 μm to about 15 μm.

32. The method of claim 28, wherein the liquid carrier comprises any one or more of a long-chain glyceride, a free fatty acid, a fatty acid ester, and a medium-chain glyceride.

33. The method of claim 32, wherein the liquid carrier comprises at least one long-chain glyceride, and the at least one long-chain glyceride is selected from the group consisting of any one or more of peanut oil, soybean oil, sunflower oil, olive oil, sesame oil, colza oil, almond oil, safflower oil, corn oil, linseed, oil, rapeseed oil, evening primrose oil, grape seed oil, cottonseed oil, flaxseed oil, and menhaden oil.

34. The method of claim 33, wherein the liquid carrier comprises at least one fatty acid ester, and the at least one fatty acid ester is selected from the group consisting of glycerol mono-, di, or tri-esters of a long or medium chain fatty acid.

35. The method of claim 33, wherein the liquid carrier comprises at least one fatty acid ester, and the at least one fatty acid ester is selected from the group consisting of propylene glycol mono- and di-esters of a long or medium chain fatty acid.

36. The method of claim 28, wherein the liquid carrier comprises at least one phospholipid.

37. The method of claim 36, wherein the phospholipid is lecithin.

38. The method of claim 2, wherein the liquid carrier comprises at least one lipophilic surfactant.

39. The method of claim 2, wherein the mixture comprises from about 4% to about 80% progesterone by weight of the mixture.

40. The method of claim 2, wherein the mixture comprises from about 10% to about 70% progesterone by weight of the mixture.

41. The method of claim 2, wherein the mixture comprises from about 20% to about 60% progesterone by weight of the mixture.

42. The method of claim 2, wherein the mixture comprises from about 40% to about 50% progesterone by weight of the mixture.

43. The method of claim 2, wherein the mixture comprises from about 20% to about 96% liquid carrier by weight of the mixture.

44. The method of claim 2, wherein the mixture comprises from about 30% to about 90% liquid carrier by weight of the mixture.

45. The method of claim 2, wherein the mixture comprises from about 40% to about 80% liquid carrier by weight of the mixture.

46. The method of claim 2, wherein the mixture comprises from about 50% to about 60% liquid carrier by weight of the mixture.

47. The method of claim 2, wherein the mixture comprises from about 0.05% to about 5% phospholipid and/or lipophilic surfactant by weight of the mixture.

48. A wet-milled progesterone composition prepared according to the method of claim 28.
